(19) United States
(12) Patent Application Publication
(10) Pub. No.: US 2025/0314613 A1
(43) Pub. Date: Oct. 9, 2025

(54) PACKAGED SEMICONDUCTOR SENSOR WITH AN OPEN DETECTION SURFACE AND SEALED DETECTION WELL

(71) Applicant: DiagMetrics, Inc., Madison, CT (US)

(72) Inventor: John James Daniels, Madison, CT (US)

(21) Appl. No.: 18/628,747

(22) Filed: Apr. 7, 2024

(51) Int. Cl. G01N 27/414; G01N 33/543; H01L 23/00; H01L 23/498

(52) U.S. Cl. CPC G01N 27/4145; G01N 33/5438; H01L 23/4985; H01L 24/29; H01L 24/32; H01L 2224/2919; H01L 2224/32225

(57) ABSTRACT

The present invention relates to a novel packaging structure for a bare die semiconductor sensor, specifically designed for diagnostic applications where direct fluid sample contact with the sensor's detection area is required. The packaged semiconductor sensor comprises a semiconductor die with a top surface featuring bond pads and a detection area, and a bottom surface. A support member with a top side, a bottom side, and a detection window forms a sample well for receiving a fluid sample when aligned with the detection area. Z-axis conductive adhesive electrically connects bond pads to conductive traces on the support member. A sealing member seals the sample well, preserving sensor functionality while allowing the detection area exposure to the sample. This innovative packaging solution protects the sensor from environmental factors and maintains electrical integrity, enabling accurate and efficient biomarker detection in diagnostic procedures.

### BACKGROUND

This section is intended to provide a background or context to the exemplary embodiments of the invention as recited in the claims. The description herein may include concepts that could be pursued, but are not necessarily ones that have been previously conceived, implemented or described. Therefore, unless otherwise indicated herein, what is described in this section is not prior art to the description and claims in this application and is not admitted to be prior art by inclusion in this section.

The present invention relates generally to semiconductor sensors and, more particularly, to a unique packaging structure and manufacturing process for a bare die semiconductor sensor used in diagnostic and environmental applications, where a portion of the bare die sensor must remain exposed to allow a sample to be received at a detection area of the sensor.

In the field of semiconductor devices, including resistors, transistors, diodes, capacitors, and integrated circuits, the standard packaging approach is to fully encapsulate the semiconductor die to safeguard the internal semiconductor materials and device features. Traditional packaging solutions provide electrical connectivity through wire bonding that connects device features on the die with external pins or leads, which can then soldered onto a printed circuit board (PCB) or connected through a socket that is soldered on the PCB. This full encapsulation is considered essential for protecting the sensitive components from environmental factors that could compromise their integrity and functionality.

However, such packaging techniques are not optimal for semiconductor sensors designed to interact with a fluid sample for the detection of various analytes. These sensors require direct exposure of certain areas or the semiconductor device to the sample while still maintaining the integrity of the electrical connections and the sensor itself. There exists a need for a packaging structure that allows the sensor to function effectively in its intended diagnostic application.

Conventional packaging technologies face significant challenges when applied to diagnostic semiconductor sensors. Full encapsulation restricts access to the active area of the sensor device, preventing the necessary interaction with the fluid sample. This limitation has prompted the need for a new packaging approach that both protects the device and allows the fluid sample to contact the detection area of the sensor.

Moreover, the increasing demand for rapid, accurate, and point-of-care diagnostic tools necessitates the development of semiconductor sensors that can be incorporated into compact and user-friendly devices, which is impeded by traditional packaging methodologies.

### BRIEF SUMMARY

The invention described herein addresses these challenges by providing a packaged semiconductor sensor with a novel structure that includes an open detection area for fluid sample contact. This configuration enables the semiconductor sensor to analyze samples effectively while ensuring that the rest of the semiconductor die is adequately protected and the electrical connections are maintained.

In accordance with an aspect of the invention, a packaged semiconductor sensor includes a semiconductor die having a top surface and a bottom surface, with at least two bond pads and at least one detection area located at the top surface. A support member has a top side and a bottom side, and a detection window provided as an opening in the support member from the top side to the bottom side. The opening/detection window in the support member and the detection area located at the top surface of the semiconductor die define a sample well for receiving a sample to be tested by the packaged semiconductor sensor. A least two conductive traces are provided on the bottom side of the support member. A z-axis conductive adhesive bonds and electrically connects a respective one of the bond pads to a corresponding one of the conductive traces. A sealing member seals the bottom side of the support member with the top surface of the die to seal the sample well. The z-axis conductive adhesive can also be used to form the sealing member. Alternatively or additionally, the sealing member can comprise at least one of an epoxy, glue, pressure sensitive adhesive and gasket.

In accordance with another aspect of the invention, a biosensor card assembly includes a bare die semiconductor sensor with a top surface including two or more bond pads and at least one detection area. A support member has at least a corresponding number of conductive traces as the bond pads on the bare die. The conductive traces are provided on at least a bottom side of the support member for connecting with the bond pads of the bare die. The support member has a through-hole detection window aligning with the detection area of the bare dic. A conductive adhesive is provided between each bond pad of the bare die and a corresponding conductive trace of the support member. The conductive epoxy provides an electrical connection between a respective bond pad and a corresponding conductive trace.

In accordance with another aspect of the invention, a sensor card assembly is configured for enhanced fluid sample analysis. A sensor element is provided with a detection area and a plurality of bond pads on a top surface. The support member includes a top side with integrated liquid detection features and a bottom side providing conductive traces corresponding to the bond pads. A z-axis conductive adhesive is provided between each bond pad and a corresponding conductive trace for selective electrical connection in the z-axis direction. A detection window on the top side of the support member is aligned with the detection area of the sensor element to form a sample well.

In accordance with another aspect of the invention, an integrated biosensor card and bare die sensor assembly is provided for targeted biomarker detection. A semiconductor die has a top surface with a least one sensor device and at least one sensor area and bond pads associated with each sensor device. A support member having a bottom side with conductive traces corresponding to the bond pads supports the semiconductor die and connects the semiconductor devices of the die to a printed circuit board. An accumulator in fluid communication with the sensor areas applies an electrostatic field to a fluid sample for aligning target biomarkers within a fluid sample. The accumulator facilitates enhanced detection by modulating the orientation and proximity of target biomarkers to the immobilized capture molecules at the sensor areas.

### BRIEF DESCRIPTION OF THE SEVERAL VIEWS OF THE DRAWINGS

FIG. 1 is a partial exploded perspective view of the biosensor card components, illustrating the spatial relationship between the conductive traces, the z-axis conductive adhesive, and the bare die semiconductor sensor.

FIG. 2 illustrates a top-down view of the biosensor card, showing the device features on the top surface of the bare die semiconductor sensor.

FIG. 3 is an exploded view diagram showing the layered construction of the biosensor card showing the separate components starting from the top overlay, with a liquid detection window, followed by the top conductive traces, the detection window on the support member, and the bottom conductive traces through to the bottom overlay.

FIG. 4 is a top-down, layer-by-layer view of the biosensor card's material stack.

FIG. 5 illustrates the material composition and stack-up dimensions for an etched copper flex circuit version of the biosensor card.

FIG. 6 shows a top view of a bare die semiconductor sensor, showing the layout of individual source and gate connections for four sensor devices, with a common drain.

FIG. 7 is a cross-sectional view of a GaN biosensor, showing the layered structure with a detection well at the top surface.

FIG. 8 shows top and bottom views of the fully assembled biosensor card.

FIG. 9 illustrates a semiconductor biosensor with six individually addressable GaN HEMT (High Electron Mobility) sensor devices sharing a common gate and drain.

FIG. 10 shows an alternative semiconductor sensor design with five functional test devices and one reference device.

FIG. 11 shows the die attachment process for a bare die semiconductor sensor to the support member.

FIG. 12 shows an isolated and partial assembly view of an embodiment of the biosensor card showing the arrangement of conductive traces, detection areas, sample wells, and other relevant structural features of a biosensor card assembly with four individually addressable and functionalizable sensor devices on a single bare die.

FIG. 13 illustrates an embodiment of a completed biosensor card with four individually addressable sensor devices.

FIG. 14 shows the top side and bottom side of a biosensor card with four independently functional sensor devices, each with its own sample well for targeted molecule detection within the same fluid sample.

FIG. 15 is an isolated view showing a bare die sensor attached to conductive traces.

FIG. 16 shows a biosensor card with attached sensor die, microfluidic liquid sample flow path and compressed cellulose wick, where the biosensor card assembly is electrically and mechanically connected with a printed circuit board.

FIG. 17 is a close-up view of a bare die sensor attached to a biosensor card with the detection window of the biosensor card aligned with the detection area of the sensor.

FIG. 18 shows a semiconductor sensor device with an accumulator to selectively position target molecules in a fluid sample for enhanced biomarker detection.

### DETAILED DESCRIPTION

Below are provided further descriptions of various non-limiting, exemplary embodiments. The below-described exemplary embodiments are separately numbered for clarity and identification. This numbering should not be construed as wholly separating the below descriptions since various aspects of one or more exemplary embodiments may be practiced in conjunction with one or more other aspects or exemplary embodiments. That is, the exemplary embodiments of the invention, such as those described immediately below, may be implemented, practiced or utilized in any combination (e.g., any combination that is suitable, practicable and/or feasible) and are not limited only to those combinations described herein and/or included in the appended claims.

The foregoing and other aspects of exemplary embodiments of this invention are made more evident in the following Detailed Description, when read in conjunction with the attached Drawing Figures, wherein:

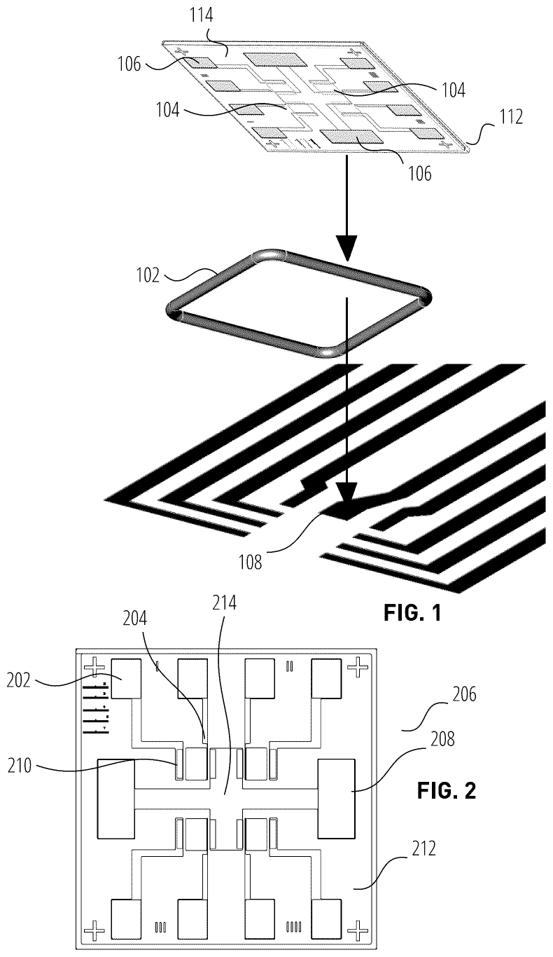

FIG. 1 shows a partial exploded view of a semiconductor biosensor card, showing the assembly and arrangement of the main conductive components. An exemplary embodiment comprises a sealing member 102, sensor devices 104, bond pads 106, conductive traces 108, a bottom surface 112, and a top surface 114.

Positioned to be attached to the bottom conductive traces is the semiconductor dic. This bare die includes GaN HEMT, g-FET, or other semiconductor elements that provide a biological or environmental sensor for the detection of targeted analytes. The top surface of the semiconductor die includes the bond pads and the detection area. The bond pads are connected to the conductive traces via a z-axis conductive adhesive, which provides both mechanical attachment and electrical continuity. In an exemplary embodiment, the z-axis conductive adhesive also provides a sealing member the seals a sample well defined by the top surface of the die and the walls of the detection window. Alternatively, additional layers can be formed to increase the volume of the sample well. Also, alternatively, the sealing member can be formed using an additional bead of a non-conductive material such as silicone.

The z-axis conductive adhesive allows for vertical (z-axis) electrical connection from the bond pads on the semiconductor die to the conductive traces on the support member, without shorting between conductive traces and/or bond pads. The z-axis conductive adhesive has an anisotropic conductivity profile that prevents lateral electrical connectivity, providing signal transmission from the detection area while also providing a reliable electrical bond, mechanical attachment and a fluid seal, without interfering with the detection area on the top surface of the die. An example of a z-axis conductive adhesive is an anisotropic conductive adhesive 125-01A/B-187 from Creative Materials, Ayer, MA. The conductive adhesive is applied, for example, using a conventional die bonder semiconductor processing equipment so that after dispensing the adhesive onto the conductive traces and a pick and place operation, the adhesive is between each bond pad of the bare die and corresponding conductive trace of the support member.

The packaged semiconductor sensor includes a semiconductor die having a top surface and a bottom surface, with at least two bond pads and at least one detection area located at the top surface. A support member has a top side and a bottom side, and a detection window provided as an opening in the support member from the top side to the bottom side. The opening/detection window in the support member and the detection area located at the top surface of the semiconductor die define a sample well for receiving a sample to be tested by the packaged semiconductor sensor. A least two conductive traces are provided on the bottom side of the support member. Each bond pad of the bare die is aligned with and electrically connected to a respective conductive trace via the z-axis conductive epoxy without the need for wire bonding.

A z-axis conductive adhesive bonds and electrically connects a respective one of the bond pads to a corresponding one of the conductive traces. A sealing member seals the bottom side of the support member with the top surface of the die to seal the sample well. The z-axis conductive adhesive can also be used to form the sealing member. Alternatively or additionally, the sealing member can comprise at least one of an epoxy, glue, pressure sensitive adhesive and gasket.

The detection window/opening and the detection area collectively define a sample well for receiving a fluid sample to be analyzed. A sealing member can be integrated or formed separately with the z-axis conductive epoxy that seals the sample well. For example, the sealing member can be composed of the z-axis conductive epoxy, and/or the sealing member can be composed of a non-conductive adhesive that seals the detection area and detection window to form the sample well and provide a barrier to protect the z-axis conductive adhesive from contacting a fluid disposed in the sample well.

FIG. 2 shows a top-down view of a semiconductor biosensor, detailing the surface features integral to the functionality as a sensor including features at the top surface that are involved in the detection of specific analytes. An exemplary embodiment comprises a bond pad 202, a gate 204, a die 206, a drain 208, a source 210, and a top side 212. At the center of the image is the detection area, a key functional part of the biosensor where the capture molecules are immobilized for selective binding and detection of target molecules present in a fluid sample. The capture molecules at the detection area interact with the fluid sample directly and therefore the detection area at the top surface of the packaged semiconductor sensor must remain open for receiving the sample.

Surrounding the detection area are bond pads, which are small, conductive areas on the top surface of the bare die. Each bond pad is positioned to interface with corresponding conductive traces on the support member to provide the transmission of changes in electrical signals resulting from the detection events (capture molecule/target molecule binding) that occurs at or near the detection area the top surface of the die.

The sensor devices on the bare die includes gate, source and drain features connected with the bond pads. The layout of the bare die features are selected to enable the electrical connections to the conductive traces via the z-axis conductive adhesive while ensuring the detection area remains unobstructed for sample interaction.

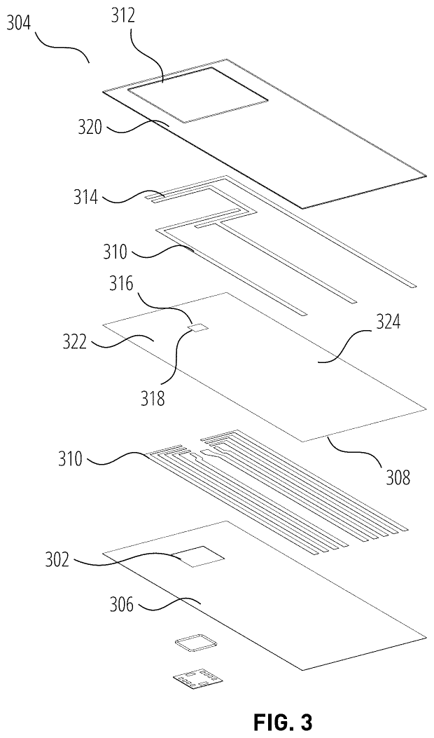

FIG. 3 shows an exploded view of the layered structure of the biosensor card, providing a detailed illustration of each layer and its respective components as they would be assembled in the manufacturing process. An exemplary embodiment comprises a bare die window 302, a biosensor card 304, a bottom overlay 306, a bottom side 308, a conductive trace 310, a detection window 312, liquid detection features 314, a liquid detection window 316, an opening 318, a top overlay 320, a support member 322, and a top side 324.

A top overlay is the uppermost layer of the biosensor card. Directly beneath the top overlay are the conductive traces that form liquid detection features, in this exemplary embodiment the conductive traces are etched copper on a flexible substrate material such as Kapton. At least one liquid detection feature is integrated into the top surface of the support member to monitor presence of a fluid sample.

A detection window is formed as an opening within the support member. The detection window aligns with the detection area of the semiconductor die, enabling the fluid sample to contact the biosensor's detection region.

The bottom side of the support member has another set of conductive traces. These traces on the bottom side provide electrical connections to the corresponding bond pads on the semiconductor die.

Starting from the top of the figure, the first layer is the top overlay, which functions as a protective cover for the underlying components. This overlay features a liquid detection window that allows the liquid sample to reach the liquid detection features. The liquid detection features are used for determining the flow of a liquid sample before and after the sample is received by the detection area of the biosensor.

Below the top overlay, the top conductive traces are shown. The conductive traces can be etched copper or printed conductive ink and provide the electrical pathways on the flexible substrate support member to provide electrical connectivity to the sensor elements.

The support member layer includes a detection window, an aperture that is aligned with the detection area on the semiconductor die and defines along with the detection area the sample well where the fluid sample is collected and analyzed.

The support member provides a platform onto which the semiconductor die will be attached. The support member includes bottom conductive traces on its reverse side, which are in turn connected to external electronic circuitry and provide a means for electrical signals to be read out from the sensor.

A bottom overlay protects the bottom side of the biosensor card, completing the assembly. The bottom overlay includes a bare die window that provides an opening to the bottom conductive traces for connecting with the bond pad of the bare die via the z-axis conductive adhesive.

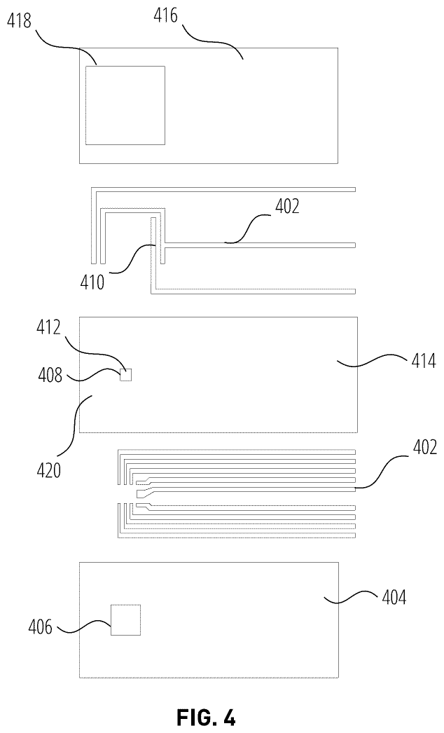

FIG. 4 shows a layered breakdown of the biosensor card, depicted in a top-down view. This drawing illustrates each component of the material stack to indicate the layout and individual function of the layers that form the complete biosensor card. An exemplary embodiment comprises a conductive trace 402, a bottom overlay 404, a bare die window 406, a detection window 408, a liquid detection features 410, an opening 412, a support member 414, a top overlay 416, a liquid detection window 418, and a top side 420.

The topmost layer shown is the top overlay, which acts as the protective outer covering of the biosensor card. It features a predefined liquid detection window that corresponds to the location of the underlying liquid detection features. The liquid detection features are formed on the top side and operative to indicate a fluid sample presence and flow characteristics. Below the top overlay is the layer of top conductive traces that form the liquid detection features.

The support member has the conductive traces formed thereon either by a subtractive process, such as etching copper foil, or by an additive process, such as screen-printing conductive silver ink. The support member also includes the detection window that aligns with the detection area on the semiconductor die, allowing the biosensor to interact with the test sample.

Beneath the support member, bottom conductive traces connect the die sensor devices features with an external electronic circuit. The bottom overlay protects and insulates the bottom conductive traces and includes the bare die window for connecting the bare die with the conductive traces.

The support member can be one of a flex circuit having an etched metal pattern forming the conductive traces, a plastic substrate having printed conductive ink forming the conductive traces, and rigid circuit board having at least one of etched metal and printed conductive ink forming the conductive traces.

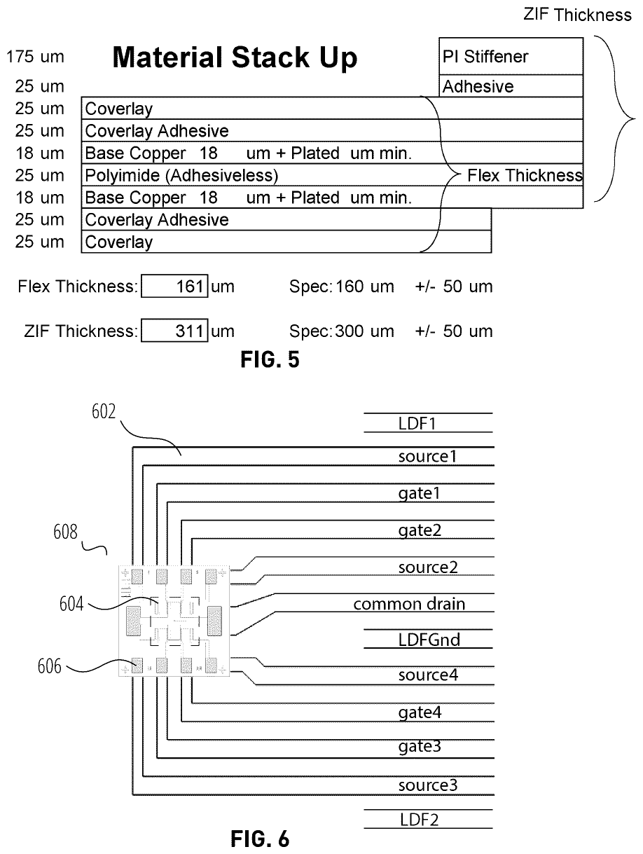

FIG. 5 shows the material stack-up for a biosensor card, specifically designed in this exemplary embodiment as an etched copper flex circuit. This drawing illustrates the various layers and their respective thicknesses for forming the biosensor card using conventional and well-known manufacturing processing and materials. The materials and thickness as for example only, other constructions can also be used. For example, a polyester (PET) or other suitable plastic substrate may be used with screen printed conductive ink forming the conductive traces. This plastic substrate embodiment may be particularly advantageous since the additive manufacturing process will be lower cost and have less environmental impact that the use of an etched copper flex circuit construction. The die attach process can be done at relatively lower temperatures, for example, using a UV curable z-axis conductive adhesive or tape, making the lower cost plastic substrate with screen printed conductive ink an attractive alternative to etch copper on Kapton.

The stack-up begins with the top overlay or coverlay, a protective layer measuring 25 micrometers (μm) in thickness that protects the underlying circuitry. A layer of overlay adhesive of the same thickness secures the top overlay to the base copper layer and the support member. The base copper layer, which is 18 μm thick, forms the conductive pathways for the circuit and includes additional thickness from plating, ensuring robust electrical connections.

Following the copper layer, a 25 μm thick adhesiveless polyimide layer forms the substrate of the support member. Another etched copper layer of 18 μm plus plating forms the bottom conductive traces.

To complete the stack, an additional overlay adhesive and a bottom overlay layer, each 25 μm thick, help to encapsulate and protect the entire assembly. For dimensional stability and to interface with an electronic circuit connector, such as ZIF connector, a stiffener with adhesive is incorporated at the edge of the biosensor card, contributing to a total ZIF connector end thickness of 311 μm.

FIG. 6 shows a detailed top view of a multi-sensor GaN biosensor device with four individually addressable sensor elements, each with its own source and gate connections and sharing a common drain. The drawing illustrates the design and layout of the sensor's ohmic features that form the bond pads on the top surface of the bare die. An exemplary embodiment comprises a conductive trace 602, a detection area 604, a bond pad 606, and a die 608.

A detection area is formed at the central region of the die but could be formed at other locations and the die could also include other electronic features connected with features of the sensor devices. For example, resistor, capacitors, transistors and other semiconductor electronic devices can be provided directly on the die and/or provided as discrete electronic devices provided on a printed circuit board connected with the sensor devices through the biosensor card. Capture molecules are immobilized at the detection area that are specific to the target analyte(s). The detection area is positioned to be in direct contact with the sample in the detection well.

Surrounding the detection area, the bare die features various bond pads, which serve as the terminals for electrical connectivity. The bond pads are connected via the z-axis conductive adhesive to the conductive traces and form the electrical pathways for signal detection.

The conductive traces are laid out to provide electrical connections to the designated source and gate terminals for each of the four sensor devices provided on the exemplary bare dic. These traces are labeled for clarity, with “sourcel” through “source4” indicating the source connections for each sensor and “gatel” through “gate4” for the gate connections. The “common drain” trace provides a streamlined and simplified electronic circuit.

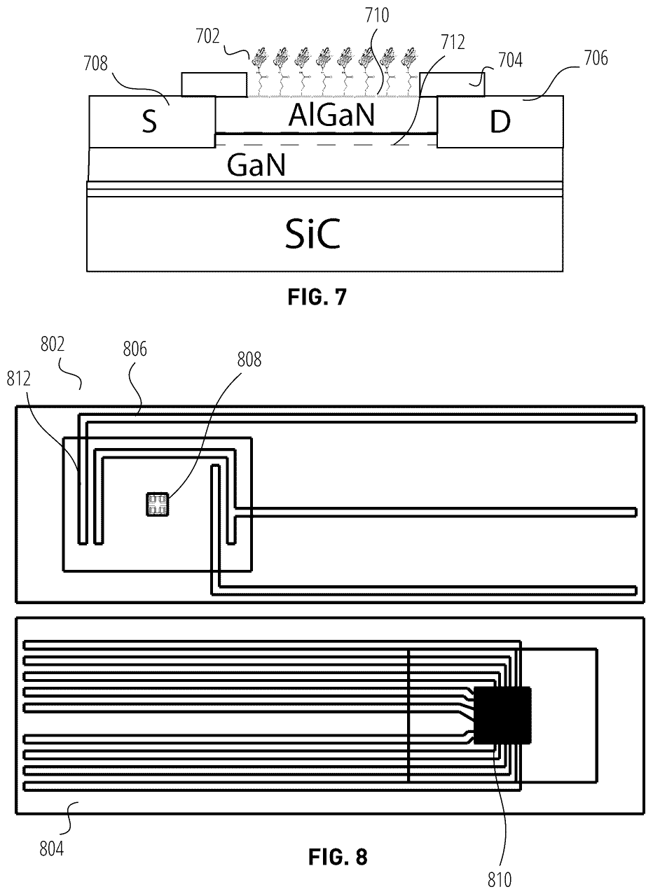

FIG. 7 provides a cross-sectional representation of a Gallium Nitride (GaN) biosensor, illustrating the essential components and their arrangement within the device. This detailed depiction is instrumental for understanding the biosensor's functional design, particularly its capability to detect target molecules. An exemplary embodiment comprises capture molecules 702, a sample well 704, a drain 706, a drain 708, a detection area 710, and a 2DEG 712.

In this exemplary embodiment, the substrate of the structure is a Silicon Carbide (SIC) wafer. Above the SiC substrate, the primary functional layer of the GaN is formed. Other wafer substrates are available for GaN HEMT fabrication, such as sapphire. The GaN layer has the advantage of wide bandgap properties that facilitate high electron mobility and contribute to the sensor's sensitivity and response time. Within this GaN layer, a two-dimensional electron gas (2DEG) channel forms naturally at the interface with the AlGaN layer. The 2DEG is a thin layer of mobile electrons that is highly sensitive to changes in electric fields and charge density.

The Aluminum Gallium Nitride (AlGaN) layer works in conjunction with the underlying GaN to create the 2DEG channel. The material properties of AlGaN, including its adjustable bandgap and electron mobility, can be finely tuned during manufacturing to optimize the sensor's performance for specific requirements.

The detection area is formed at the surface of AlGaN layer. This area is where capture molecules are immobilized. The capture molecules are designed to selectively bind to specific analytes, initiating a change in the electrical properties of the 2DEG below, which can then be measured and translated into a detectable signal indicating the presence of the target substances.

Directly above the detection area, a detection well is provided. This well is where the sample containing potential target molecules will interact with the capture molecules.

The GaN HEMT sensor can be improved through the systematic optimization of features formed at the wafer level balanced with materials and processes in the fabrication of the functionalized sensor devices of the die attached to the biosensor card connected with the reader electronics PCB. For example, thinning the AlGaN layer in a GaN HEMT sensor has the potential to increase the sensor's sensitivity, as it brings the detection area—where the capture molecule/target molecule binding occurs-closer to the 2DEG. The proximity can enhance the perturbation effect of the bound molecules on the 2DEG, potentially leading to a stronger modulation of the channel's conductivity when a target molecule binds to a capture molecule. This closer interaction means that even small changes at the surface can have a more significant impact on the 2DEG channel, potentially leading to improved sensitivity of the biosensor. The thickness of the AlGaN layer in a GaN HEMT (High Electron Mobility Transistor) device is an important design parameter. In the context of biosensing applications, there's a trade-off to consider: a thinner AlGaN layer can indeed bring the detection area closer to the 2DEG, potentially increasing the sensor's sensitivity, but it can also introduce several challenges.

For example, the AlGaN layer typically ranges from a few nanometers to tens of nanometers. The optimal thickness is often a result of empirical optimization and depends on the specific application. For biosensing, it might be thinned to just above the critical thickness that prevents the introduction of dislocations and other crystal defects.

A thinner AlGaN layer can alter the electrical properties of the HEMT structure. It can affect the 2DEG density and the device's threshold voltage. These properties should remain within certain limits to maintain the device's operational integrity, so part of the systematic improvement of the bare die structure can include a design of experiments aimed at the optimization of the AlGaN layer, and may include an insulator stack comprising different materials that provide the best balance of fabrication costs, biosensor sensitivity and device robustness to withstand all the fabrication processes, shelf life considerations and test performance.

For example, making the AlGaN layer too thin could compromise the structural integrity of the device, potentially leading to mechanical instability or breakdown under stress or during handling. Thinning the AlGaN layer could also increase the likelihood of leakage currents, especially if the layer becomes too thin to effectively confine the electrons in the 2DEG. This could lead to increased noise and decreased signal-to-noise ratio, adversely affecting sensor sensitivity. The thinner the layer, the more difficult it can be to manufacture it with consistent quality and across large wafers. Non-uniformities in thickness can result in device-to-device variability, affecting yield and performance. If the AlGaN layer is made too thin, direct tunneling may occur, where charge carriers tunnel through the AlGaN barrier instead of being properly controlled by the gate. This could severely degrade device performance.

In general, the design and fabrication of GaN HEMT devices will involve a balancing act where the AlGaN layer is made as thin as possible to enhance sensitivity, but not so thin that it induces significant leakage currents, decreases electron mobility, or adversely impacts the 2DEG characteristics.

FIG. 8 illustrates the fully assembled biosensor card as viewed from both the top and bottom perspectives.

The top view photo shows the liquid detection features formed from the top conductive traces. The liquid detection features are useful for determining the flow of the liquid sample just before and after the sample passes over the biosensor.

In the bottom view, the bare die semiconductor sensor is shown mechanically fixed on the biosensor card and electrically connected to the bottom conductive traces via a z-axis conductive adhesive. This anisotropic adhesive material provides a reliable bond that maintains the electrical integrity of the connection while allowing for electrical conduction only in the vertical (z-axis).

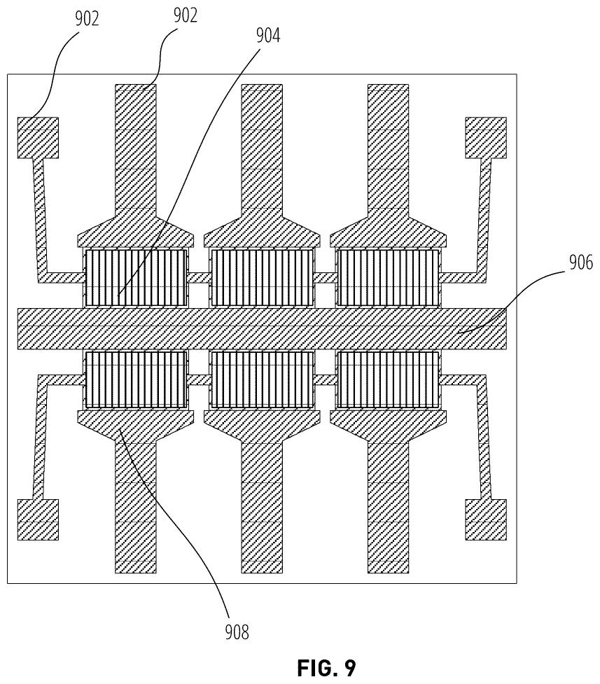

FIG. 9 shows an alternative design of a semiconductor biosensor die with an array of six Gallium Nitride (GaN) High Electron Mobility Transistor (HEMT) devices. In this embodiment, the gate and drain are shared features with individual addressability of each sensor device provided through the individual connection to each source.

An exemplary embodiment comprises a bond pad 902, a detection area 904, a drain 906, and a source 908. That is, the sensor devices each have its own source electrode, while sharing a common gate and drain. This configuration that facilitates the parallel and/or serial readout of test results for multiple biomarkers. This shared structure also reduces the complexity of the biosensor card and enhances the die's compactness, an important consideration since die cost is typically relatable to die size.

Each source bond pad connects the individual sensor devices and is linked to a separate detection area for each sensor device. These separately addressable detection areas are functionalized with unique capture molecules to enable the detection of various biomarkers, or the same capture molecule can be provided and an average reading from each addressable sensor device taken as the test output. This multi-sensor approach allows for a broad spectrum of diagnostic capabilities, such as simultaneous testing for different viral proteins or pathogens.

In application, this sensor array can effectively analyze complex biological samples, identifying the presence of multiple biomarkers with high specificity and sensitivity. For instance, each sensor device could be functionalized to detect distinct biomarkers associated with various diseases or conditions, offering a comprehensive diagnostic tool within a single semiconductor dic.

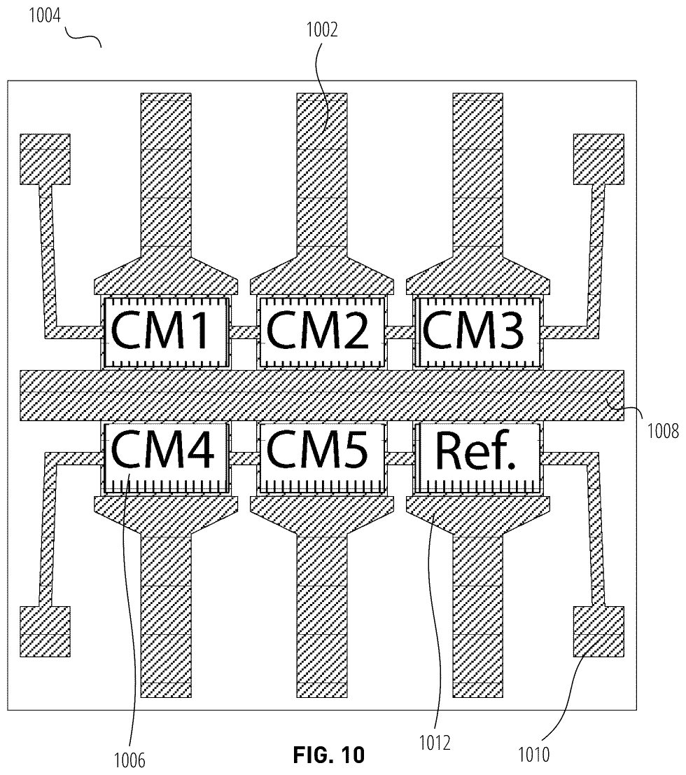

FIG. 10 shows a multi-sensor biosensor die with five test sensors and one reference sensor, all integrated onto a single bare die. Each test sensor device can be uniquely functionalized to detect a specific biomarker. An exemplary embodiment comprises a bond pad 1002, a die 1004, a detection area 1006, a drain 1008, a gate 1010, and a source 1012.

The test sensor devices, labeled CM1 (Capture Molecule 1) through CM5, are functionalized with distinct capture molecules that are selected for their high affinity to particular biomarkers, allowing the sensors to identify and measure the presence of multiple analytes such as the SARS-COV-2 N and S proteins, Flu A and B antigens, and the Respiratory Syncytial Virus (RSV).

The reference sensor device, labeled ‘Ref.’ can be used to normalize the test readings and account for environmental variables like temperature and humidity that could affect sensor performance. It is functionalized with a capture molecule that exhibits representative electrical characteristics similar to the other sensors. This feature enables the reference sensor to act as a control point, maintaining the reliability of the biosensor's output by providing a consistent baseline for comparison.

In practice, a comparator circuit may be used to contrast the signal from the reference sensor with that of each test sensor. This comparison helps to ensure that any signal variations are attributable to the presence of the target biomarker, rather than extraneous environmental factors.

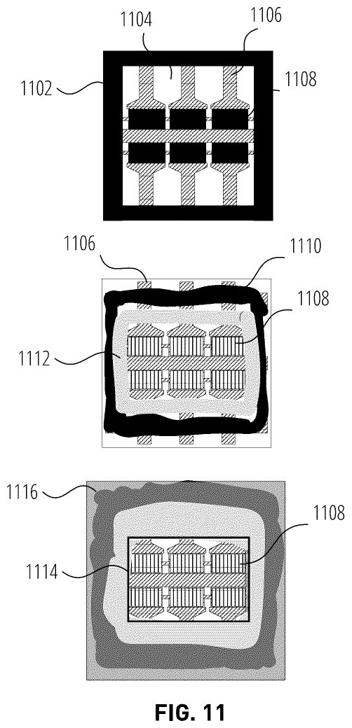

FIG. 11 shows the detailed procedure for attaching a bare die semiconductor sensor to a support member during the fabrication of the biosensor card. The bare die is attached to the support member so that the detection window in the support member aligns with the detection areas on the sensor devices. An exemplary embodiment comprises a mask 1102, a top surface 1104, a bond pad 1106, a detection area 1108, a z-axis conductive adhesive 1110, a non-conductive adhesive 1112, a liquid detection window 1114, and a support member 1116.

In the die attach operation, a z-axis conductive epoxy bead is applied around the edges of the bare die. The conductive epoxy serves multiple purposes: it secures the bare die to the support member; provides electrical connectivity between the die and the conductive traces on the support member; and seals the sample well defined the by the top surface of the bare die and the walls of the detection window in the support member. The sample well can include additional layers, such a patterned plastic or an external well structure to hold a volume of a liquid sample as a pool in contact with the detection well. As an alternative or in addition to the z-axis conductive adhesive sealing the sample well, a barrier bead of a non-conductive adhesive can be provided that prevents the z-axis conductive epoxy from being exposed to solvents and other materials or processes that could degrade the z-axis conductive adhesive. This non-conductive adhesive may be beneficial depending on the post-die attach processing of the biosensor card. For example, if a solvent used to clean the detection area prior to functionalization would dissolve the cured z-axis conductive adhesive, the non-conductive adhesive could be provided as a barrier to prevent exposure of the z-axis conductive adhesive to the solvent. As another barrier, a gasket may be pick and placed and held in place by the z-axis conductive adhesive bond or the gasket may have a better bond strength to give more flexibility to the choice of z-axis conductive material.

Following the application of the conductive epoxy in the bare die window, the bare die is placed onto the epoxy bead and the epoxy is cured. Pressure can be applied during curing to ensure good physical contact and the formation of a robust electrical and mechanical bond between the die and the support member.

Once the epoxy has cured, a protection layer can also applied on the sensor, leaving the detection areas and bond pads open. This protection layer can be formed from cooperating hydrophobic and hydrophilic structures. For example, at the wafer level or in materials adjacent to the die (e.g., on the biosensor card). During the die attach process, the bare die is placed onto the support member so that the detection window in the support member aligns with the detection area of the sensor devices. The views shown in FIG. 11 are looking from the bottom up, in the actual die attach process the support member is held on a work holder and z-axis conductive epoxy bead is formed at the bare die window of the support member, then the bare die is pick and placed onto the z-axis conductive epoxy bead and pressure applied to ensure a good electrical contact, mechanical attachment and seal. Depending on the type of z-axis conductive epoxy, the applied pressure may be held during a snap or UV curing process.

For example, the z-axis conductive adhesive comprises at least one of an anisotropically conductive epoxy, anisotropically conductive glue, and anisotropically conductive pressure sensitive adhesive film. The conductive adhesive provides at least one of an electrical connection and mechanical attachment, and the respective bond pad and corresponding conductive trace is electrically connected by the z-axis conductive adhesive without causing short circuits between adjacent or other bond pads and conductive traces. The sealing member can be a silicone adhesive or sealing material that forms a barrier between the sample well and the z-axis conductive adhesive.

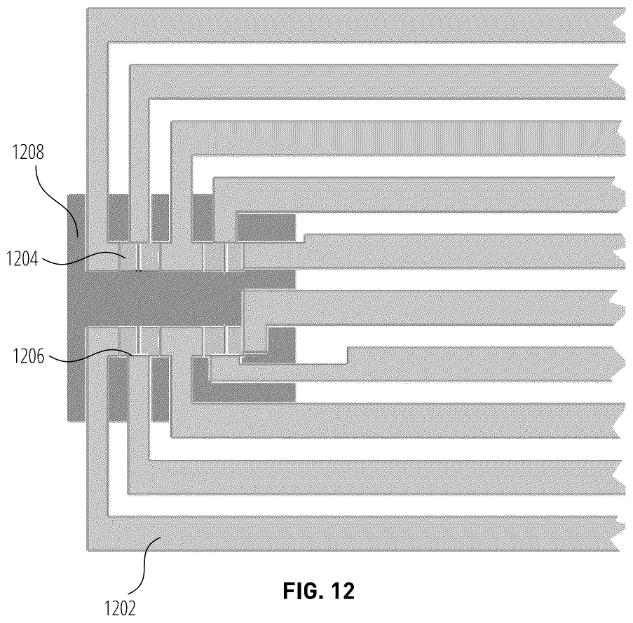

FIG. 12 shows an isolated view of the packaged semiconductor sensor where a bare die semiconductor sensor device is attached to a flex circuit biosensor card using a z-axis conductive adhesive. The view shows an isolated and partial assembly of the biosensor card showing the arrangement of conductive traces, detection areas, sample wells, and other relevant structural features of a biosensor card assembly with four individually addressable and functionalizable sensor devices on a single bare die. The exemplary embodiment comprises a conductive trace 1202, a detection area 1204, a detection window 1206, and a die 1208.

Conductive traces are patterned on the substrate of the biosensor card to provide electrical pathways to enable signal detection and transmission from the sensor elements to a data processing unit, e.g., reader electronics provided on a printed circuit board. Individually addressable detection areas and individually accessible sample wells are provided so that a fluid sample can be tested for different target analytes by each of the four individually addressable sensor devices.

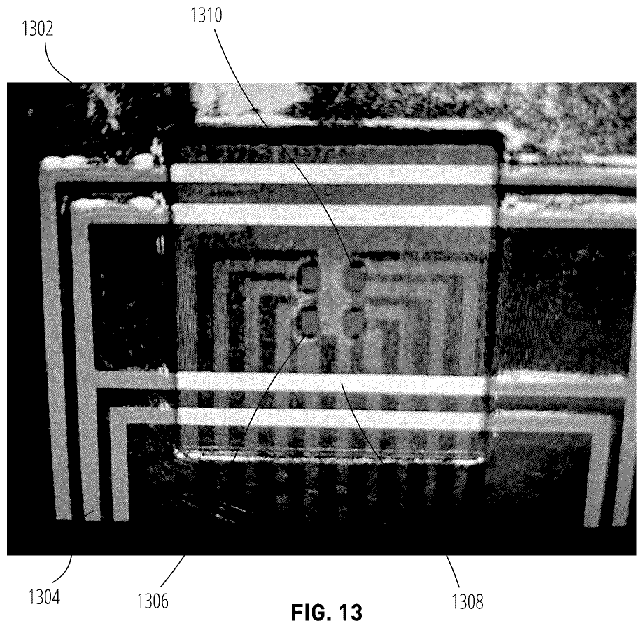

FIG. 13 shows an embodiment of a completed biosensor card with four individually addressable sensor devices. Each sensor is equipped with its own sample well, allowing for the detection of distinct target molecules from the same fluid sample. The detection areas of each of the sensor devices can be individually functionalized to perform multiplexed testing within a compact and integrated system. This configuration allows for simultaneous and selective analysis of various biomarkers in the same fluid sample.

The system comprises a biosensor card 1302, a conductive trace 1304, a detection window 1306, a liquid detection features 1308, and a sample well 1310.

The completed biosensor card is a multi-sensor platform where each sensor device has an individually assessable sample well. The detection windows, visible in the center of the photo, receive the fluid sample. The biosensor card includes liquid detection features that are used to determine the flow of the liquid sample before and after the detection areas.

This biosensor card allows for the simultaneous analysis of different target molecules within a single fluid sample by using four independently addressable sensor devices. Each of these sensor devices can be individually functionalized to detect specific biomarkers enabling multiplexed testing capabilities for scenarios where multiple assays need to be performed concurrently, such as in comprehensive medical diagnostics or complex environmental analyses.

The biosensor card assembly includes a bare die semiconductor sensor with a top surface including two or more bond pads and at least one detection area. A support member has at least a corresponding number of conductive traces as the bond pads on the bare die. The conductive traces are provided on at least a bottom side of the support member for connecting with the bond pads of the bare die. The support member has a through-hole detection window aligning with the detection area of the bare die. A conductive adhesive is provided between each bond pad of the bare die and a corresponding conductive trace of the support member. The conductive epoxy provides an electrical connection between a respective bond pad and a corresponding conductive trace.

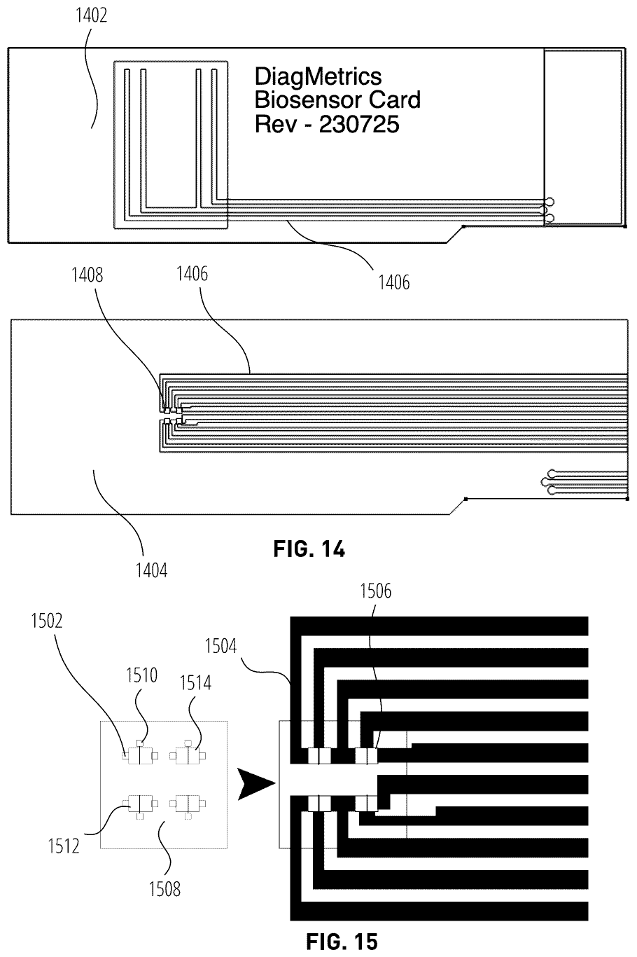

FIG. 14 presents a detailed schematic of a biosensor card, delineating various integral components and their arrangement for multiplexed biomolecular detection. The card comprises a top side and a bottom side, ensuring structural integrity and facilitating electrical connections via conductive traces. Central to the design is the detection window, which is aligned with individually accessible sample wells situated on the top side of the biosensor card.

An embodiment comprises a biosensor card 1402, a bottom side 1404, a conductive trace 1406, and a detection window 1408.

Each sensor device is independently addressable, enabling the biosensor card to analyze distinct target molecules concurrently within a singular fluid sample. This capacity for individual functionalization of the sensor devices allows for a comprehensive analysis of multiple biomarkers.

The support member can be fabricated from a flexible or rigid substrate material and the conductive traces are formed on the flexible substrate through at least one of an additive manufacturing process and a subtractive manufacturing process.

FIG. 15 shows an exemplary layout of a biosensor card with conductive traces leading to distinct sensor devices. The exemplary embodiment comprises a bond pad 1502, a conductive trace 1504, a detection window 1506, a die 1508, a gate 1510, a detection area 1512, and one or more semiconductor sensor devices 1514.

This layout enables multiplexing testing where different sensor devices on the same biosensor card can be selectively activated and read, allowing for complex diagnostic assays to be performed in parallel.

The addressable detection wells are arranged to correspond with the detection areas, and each well is configured for analyzing specific target molecules from a fluid sample introduced through the detection window. The conductive traces are patterned for independent electrical addressing of each sensor device, enabling the simultaneous and separate analysis of multiple biomarkers.

This layout enables multiplexing capability, where different sensor areas on the same card can be selectively activated and read, allowing for complex diagnostic assays to be performed in parallel. Such a configuration is key for high-throughput screening and real-time monitoring of diverse biological samples, indicating the card's potential utility in advanced medical diagnostics, environmental sensing, and bioanalytical systems.

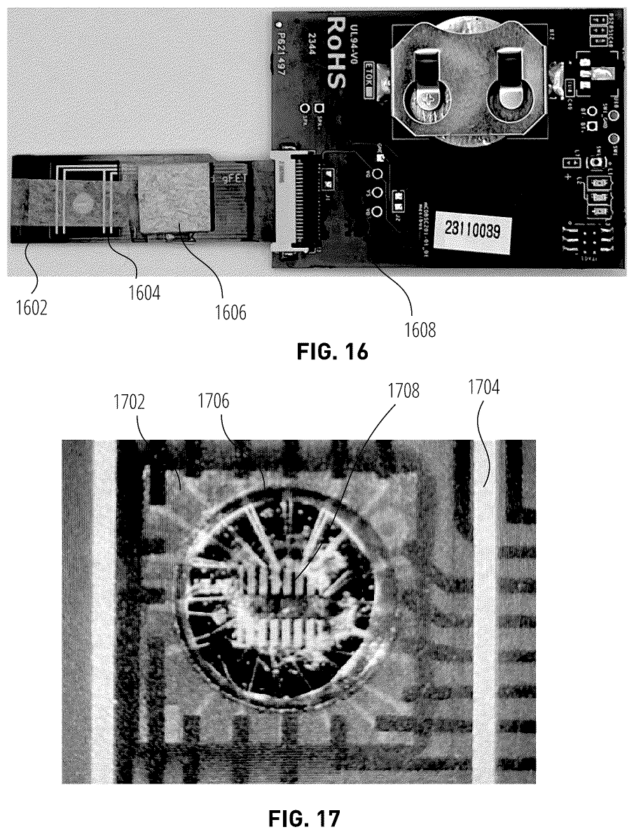

FIG. 16 shows an embodiment of the biosensor card interfaced with a printed circuit board (PCB) through a zero insertion force (ZIF) connector. The biosensor card includes microfluidic channels comprising a filter paper, guiding a fluid sample along a defined path. Initially, the sample encounters a liquid detection feature, which could be an assay or sensor that preliminarily assesses the presence or characteristics of the sample.

The sample then flows over the detection wells where the sample interacts with the detection area of the biosensor. As the sample exits the detection wells, it passes over a second detection feature. This sequential flow ensures that the sample is analyzed both before and after the primary detection event.

The compressed cellulose sponge adjacent to the microfluidic channels serves as a reservoir or wicking material to facilitate the capillary action that drives the sample through the microfluidic system without the need for external pumps.

The PCB, which the biosensor card connects to via the ZIF connector, provide electronic components to transduce the sensor signals into readable data. The support member is designed to facilitate quick-release engagement with a data processing unit via a mechanical and electrical connector interface, allowing for rapid interchangeability of the biosensor card.

FIG. 17 showcases a close-up view of a portion of a biosensor card, specifically focusing on the components for sample detection. Central to the image is a detection window, a transparent or open area that allows a fluid sample to interact with the sensor's detection area. The detection area, typically functionalized with specific reagents or biological elements, is the active site where the target molecules within the sample are captured or analyzed.

Surrounding the detection area is the detection well, which is likely designed to contain the fluid sample and ensure that it remains over the detection area for a sufficient period, enhancing the sensor's ability to detect the target molecules present.

The arrangement suggests that the detection window facilitates the introduction of the sample, the detection well serves to confine the sample over the detection area for analysis, and the detection area itself contains the elements for the biochemical interaction or reaction that leads to the detection of specific biomolecules. This setup is integral for various applications, including medical diagnostics, environmental monitoring, or any field requiring precise molecular detection.

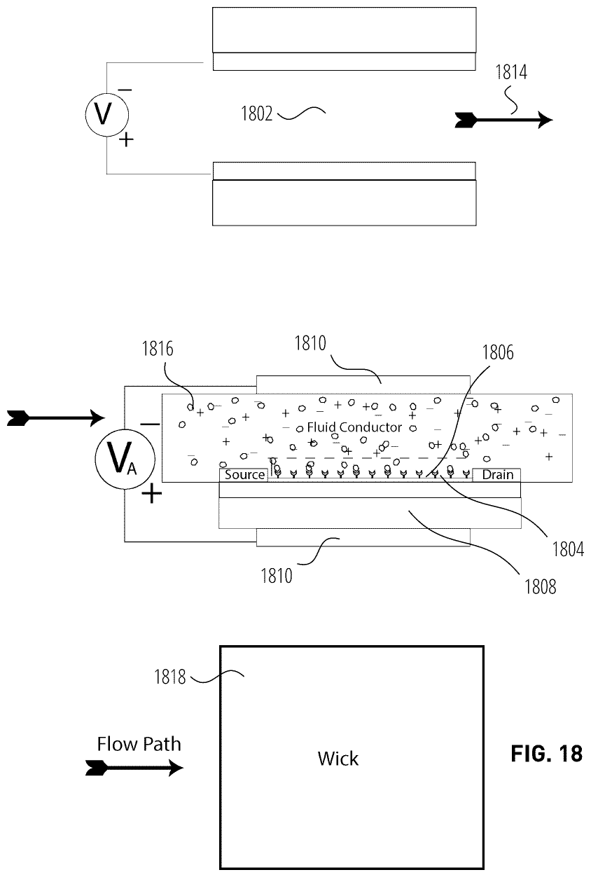

FIG. 18 shows an electrostatic control system for use with a semiconductor sensor to enhance the detection and analysis of target molecules in a fluid sample. Driving electrodes create a controlled electrostatic field across the sample flow path. The electrostatic field modulates the motion and orientation of charged particles and polar molecules within the sample. The electrostatic field can be controlled at various locations along the sample flow path so that when the sample flow passes over the detection area, the target molecules are better positioned for binding with the immobilized capture molecules. An exemplary embodiment comprises an accumulator 1802, capture molecules 1804, a detection area 1806, a die 1808, driving electrodes 1210, a flow path 1814, and target molecules 1816.

An integrated biosensor card and bare die sensor assembly is provided for targeted biomarker detection. A semiconductor die has a top surface with a least one sensor device and at least one sensor area and bond pads associated with each sensor device. As shown in other Figures, a support member having a bottom side with conductive traces corresponding to the bond pads supports the semiconductor die and connects the semiconductor devices of the die to a printed circuit board. An accumulator in fluid communication with the sensor areas applies an electrostatic field to a fluid sample for aligning target biomarkers within a fluid sample. The accumulator can be provided on the biosensor card and in the flow path of the liquid sample and facilitates enhanced detection by modulating the orientation and proximity of target biomarkers to the immobilized capture molecules at the sensor areas.

A semiconductor sensor, such as a graphene field effect transistor sensor, has optimized sensitivity that is related to the Debye screening length and the distance and orientation of the capture molecule binding sites from the detection surface. To facilitate the binding interactions, an accumulator can be provided in the flow path of the liquid sample before the sample reaches the detection area. An electrostatic field selectively concentrates and orients the target molecules. The signal applied to the conductor of the accumulator can be controlled so that the momentum of the polar molecules is altered to favor the movement of the binding site of the target molecule towards the capture molecules immobilized at the detection area.

The electrostatic field selectively controls the orientation and movement of target molecules and concentrate them in the direction of the capture molecules immobilized on the detection area. The applied electrostatic driving force induces rotational movements in polar molecules, positioning them for binding and contributing the captured charges at that detection surface to influence the flow of electrons as a detected signal. By orienting the molecules correctly, the binding efficacy to these sites is increased, leading to enhanced sensitivity and specificity of the sensor. Similarly, the immobilized capture molecule can also be a polar molecule. The electrostatic field orients the capture molecules and the applied signal can be control so that the orientation is optimized for the binding interaction with the target molecules.

The flow path can include hydrophobic and hydrophilic structures formed at the wafer level that control the flow and volume of the fluid sample to improve the wetting of the detection area and form a pool of the flowing liquid sample.

This flow path layer can be formed from cooperating hydrophobic and hydrophilic structures to control the direction and rate of the liquid sample flow. For example, at the wafer level or in materials adjacent to the die (e.g., on the biosensor card), microfabricated, screen printed or otherwise formed or applied pattern of surface energy features can be made. An accumulator applies and electrostatic field to the fluid sample passing between two insulated conductors. The electrostatic field can be applied in pulse or patterns to give momentum to a polar molecule to cause rotation. For example, an on/off DC (direct current) pulse can be tuned to maximize the separation of the polar target molecule from the other ions and polar constituents in the sample.

Creating hydrophobic and hydrophilic patterns at the wafer level enables the control of the sample fluid flow using surface modifications that alter the wetting properties of specific areas on the die. These surface properties can be controlled at the microscale for directing and confining fluid samples to targeted areas, such as the detection zones in a biosensor. The hydrophobic and hydrophilic patterns can be fabricated by selectively applying these monolayers through stencil printing or microcontact printing. Plasma treatments can also be used to modify surface energy. Exposing the detection surface to an oxygen plasma can make it more hydrophilic, while a fluorocarbon plasma can make it more hydrophobic.

The hydrophilic/hydrophobic patterns can converge the liquid sample flow path to the detection area and a pattern of more and less hydrophobic/hydrophobic regions on the detection area can control the density of capture molecules immobilized during the functionalization process. The pattern can be made to facilitate wetting active regions of the detection area. Hydrophobic boundaries can also be designed around the detection area to create a microwell or pool where the sample can accumulate. A fine grid of hydrophilic boundaries on the detection area can also control both the density and uniformity of the immobilized capture molecules, and well as utilize the surface tension of the fluid sample to draw the sample into contact with hydrophobic detection area active regions bounded by the hydrophilic grid. Hydrophobic boundaries can control the size of the pool to determine the volume of the sample that interacts with the sensor to enable quantitative analysis. In general, standardized wetting and pooling can lead to more consistent sample volumes and sensor interactions, improving the reproducibility of the sensor's readings.

The accumulator is provided in fluid communication with the sensor areas, the accumulator applies an electrostatic field to the fluid sample for aligning target biomarkers within a fluid sample. The accumulator facilitates enhanced detection by modulating the orientation and proximity of target biomarkers to the sensor areas.

For a Gallium Nitride High Electron Mobility Transistor (GaN HEMT) biosensor configuration, the general principles described for the g-FET remain consistent. However, the material properties and operation of the GaN devices can be leveraged to further refine the system. GaN has a high electron mobility and a wide bandgap, which is beneficial for biosensing applications. GaN surfaces can be modified with hydrophobic and hydrophilic patterns to control fluid flow much easier than forming patterns of graphene at the microscale. The robustness of GaN allows for a variety of surface treatments that can create these patterns without compromising the integrity of the sensor. The functionalization of the GaN detection area to immobilize capture molecules can benefit from hydrophilic/hydrophobic patterning. For instance, a hydrophilic grid can attract the sample and promote even distribution across the active area, while hydrophobic regions can prevent non-specific binding, improving signal-to-noise ratios in the sensor output.

The fine-tuning of pool sizes via hydrophobic boundaries around GaN detection areas can facilitate precise quantitative analysis by controlling the volume of the sample interacting with the sensor. GaN's stability under various environmental conditions allows for such features to be implemented with high reproducibility. GaN technology is compatible with standard semiconductor microfabrication techniques. Microfabricated patterns for controlling sample flow at the wafer level can be implemented on GaN devices using conventional etching, photolithography, or newer methods like direct laser writing.

At least one of hydrophilic and hydrophobic patterns are formed on at least one of the top side of the biosensor card and the top surface of the bare die control flow and positioning of the fluid sample over the detection area. The hydrophilic and hydrophobic patterns are arranged to create microchannels that direct the fluid sample towards the detection area. The hydrophobic patterns are located around a periphery of the detection area to contain the fluid sample. A microstructured surface on the detection area can includes a combination of hydrophilic and hydrophobic regions designed to modulate sample volume and fluid dynamics for optimizing surface wetting properties of the detection area.

The foregoing description has provided by way of exemplary and non-limiting examples a full and informative description of the invention. However, various modifications and adaptations may become apparent to those skilled in the relevant arts in view of the foregoing description, when read in conjunction with the accompanying drawings and the appended claims. However, all such and similar modifications of the teachings of this invention will still fall within the scope of the non-limiting and exemplary embodiments of this invention.

Furthermore, some of the features of the various non-limiting and exemplary embodiments of this invention may be used to advantage without the corresponding use of other features. As such, the foregoing description should be considered as merely illustrative of the principles, teachings and exemplary embodiments of this invention, and not in limitation thereof.

What is claimed is:

1. A packaged semiconductor sensor, comprising:
a semiconductor die having a top surface and a bottom surface, with at least two bond pads and at least one detection area located at the top surface;
a support member having a top side and a bottom side, and having a detection window provided as an opening in the support member from the top side to the bottom side, wherein the opening in the support member and a detection area located at the top surface of the semiconductor die define a sample well for receiving a sample to be tested by the packaged semiconductor sensor, and at least two conductive traces provided on the bottom side of the support member;
a z-axis conductive adhesive for bonding and electrically connecting a respective one of the bond pads to a corresponding one of the conductive traces; and
a sealing member for sealing the bottom side of the support member with the top surface of the die to seal the sample well.

2. The packaged semiconductor of claim 1, wherein the z-axis conductive adhesive also forms the sealing member.

3. The packaged semiconductor of claim 1, where the sealing member comprises at least one of an epoxy, glue, pressure sensitive adhesive and gasket.

4. The packaged semiconductor of claim 1, where the z-axis conductive adhesive comprises at least one of an anisotropically conductive epoxy, anisotropically conductive glue, and anisotropically conductive pressure sensitive adhesive film.

5. The packaged semiconductor of claim 1, where the support member is one of a flex circuit having an etched metal pattern forming the conductive traces, a plastic substrate having printed conductive ink forming the conductive traces, and rigid circuit board having at least one of etched metal and printed conductive ink forming the conductive traces.

6. A biosensor card assembly comprising:
a bare die semiconductor sensor with a top surface including two or more bond pads and at least one detection area;
a support member having at least a corresponding number of conductive traces as the bond pads on the bare die, the conductive traces provided on a bottom side of the support member, the support member having a through-hole detection window aligning with a detection area of the bare die;
a conductive adhesive applied between each said two or more bond pads of the bare die and corresponding conductive trace of the support member, wherein the conductive epoxy provides an electrical connection between a respective bond pad and a corresponding conductive trace.

7. The biosensor card assembly of claim 6, wherein the conductive adhesive comprises a z-axis conductive adhesive wherein the conductive adhesive provides both an electrical connection and mechanical attachment, and the respective and corresponding conductive trace is electrically connected by the z-axis conductive adhesive without causing short circuits between adjacent or other bond pads and conductive traces.

8. The biosensor card assembly of claim 7, wherein the z-axis conductive adhesive comprises an anisotropic conductive adhesive material that allows for conductivity primarily in the z-axis direction.

9. The biosensor card assembly of claim 6, wherein the support member is fabricated from a flexible substrate material and the conductive traces are formed on the flexible substrate through at least one of an additive manufacturing process and a subtractive manufacturing process.

10. The biosensor card assembly of claim 6, wherein the detection window and the detection area collectively define a sample well for receiving a fluid sample to be analyzed.

11. The biosensor card assembly of claim 10, further comprising a sealing member integrated with the z-axis conductive epoxy that seals the sample well.

12. The biosensor card assembly of claim 11, where the sealing member is composed of the z-axis conductive epoxy.

13. The biosensor card assembly of claim 11, where the sealing member is composed of a non-conductive adhesive that seals the detection area and detection window to form the sample well and provide a barrier to protect the z-axis conductive adhesive from contacting a fluid disposed in the sample well.

14. The biosensor card assembly of claim 6, further comprising at least one liquid detection feature integrated into the top surface of the support member to monitor presence of a fluid sample.

15. The biosensor card assembly of claim 6, wherein each bond pad of the bare die is aligned with and electrically connected to a respective conductive trace via the z-axis conductive epoxy without the need for wire bonding.

16. The biosensor card assembly of claim 6, further comprising an accumulator in fluid communication with the sensor areas, the accumulator for applying an electrostatic field to a fluid sample for aligning target biomarkers within a fluid sample, wherein the accumulator facilitates enhanced detection by modulating the orientation and proximity of target biomarkers to the sensor areas.

17. A sensor card assembly configured for enhanced fluid sample analysis, comprising: a sensor element with a detection area and a plurality of bond pads on a top surface;
a support member featuring a top side and a bottom side providing conductive traces corresponding to the bond pads;
a z-axis conductive adhesive provided between each of the plurality of bond pads and a corresponding conductive trace for selective electrical connection in the z-axis direction; and
a detection window on the top side of the support member aligned with the detection area of the sensor element to form a sample well.

18. The sensor card assembly configured of claim 17, further comprising liquid detection features formed on the top side and operative to indicate a fluid sample presence and flow characteristics.

19. The sensor card assembly configured of claim 17, further comprising at least one of hydrophilic and hydrophobic patterns formed on at least one of the top side and the top surface to control flow and positioning of a fluid sample over the detection area.

20. The sensor card assembly configured of claim 19, wherein the at least one of hydrophilic and hydrophobic patterns are arranged to create microchannels that direct the fluid sample towards the detection area.

21. The sensor card assembly configured of claim 19, where the hydrophobic patterns are located around a periphery of the detection area to contain the fluid sample.

22. The sensor card assembly configured of claim 19, further comprising a microstructured surface on the detection area that includes a combination of hydrophilic and hydrophobic regions designed to modulate sample volume and fluid dynamics for optimizing surface wetting properties of the detection area.

23. The sensor card assembly configured of claim 17, wherein the z-axis conductive adhesive is further defined to have an anisotropic conductivity profile that prevents lateral electrical connectivity, ensuring precise signal transmission from the detection area.

24. The sensor card assembly configured of claim 17, wherein the support member is designed to facilitate quick-release engagement with a data processing unit via a mechanical and electrical connector interface, allowing for rapid interchangeability of the sensor card assembly.

25. The sensor card assembly configured of claim 17, further comprising an accumulator in fluid communication with the sensor areas, the accumulator for applying an electrostatic field to a fluid sample for aligning target biomarkers within a fluid sample, wherein the accumulator facilitates enhanced detection by modulating the orientation and proximity of target biomarkers to the sensor areas.

26. An integrated biosensor card and bare die sensor assembly for targeted biomarker detection, comprising:
a semiconductor die having a top surface with a least one sensor device having at least one sensor area and bond pads associated with each said at least one sensor device;
a support member having a bottom side with conductive traces corresponding to the bond pads; and
an accumulator in fluid communication with the sensor areas, the accumulator for applying an electrostatic field to a fluid sample for aligning target biomarkers within a fluid sample, wherein the accumulator facilitates enhanced detection by modulating the orientation and proximity of target biomarkers to the sensor areas.

27. The for targeted biomarker detection of claim 26, further comprising a z-axis conductive adhesive providing selective electrical connection between the bond pads and the conductive traces, while preventing lateral electrical shorting.

28. The for targeted biomarker detection of claim 26, further comprising a detection window formed in the support member and aligned with the sensor areas to define at least one individually accessible sample well.
